(19) United States
(12) Patent Application Publication
(10) Pub. No.: US 2023/0097612 A1
(43) Pub. Date: Mar. 30, 2023

(54) METHOD FOR COMPUTATIONAL CONSTRUCTION OF PEPTIDE SEQUENCES

(71) Applicant: University of South Alabama, Mobile, AL (US)

(72) Inventors: Thomas Duane Johnsten, JR., Fairhope, AL (US); Aishwarya Prakash, Mobile, AL (US); Grant Thomas Daly, Satsuma, AL (US); Ryan Gene Benton, Mobile, AL (US)

(21) Appl. No.: 17/955,632

(22) Filed: Sep. 29, 2022

Related U.S. Application Data
(60) Provisional application No. 63/250,330, filed on Sep. 30, 2021.

(51) Int. Cl. G16B 30/00; G16B 15/20; C07K 1/00

(52) U.S. Cl. CPC G16B 30/00; G16B 15/20; C07K 1/00

(57) ABSTRACT

A computational method for constructing a synthetic peptide sequence is disclosed. The method of the present invention includes the steps of (i) identifying a candidate sequence building block set comprising candidate sequence building blocks from a base set comprising known functional peptide sequences and optionally known non-functional peptide sequences; (ii) selecting a qualified sequence building block set comprising qualified sequence building blocks from said candidate sequence building block set; said qualified sequence building blocks satisfying a threshold requirement and (iii) assembling said qualified sequence building blocks to generate a synthetic peptide sequence. A synthetic peptide sequence and a functional synthetic peptide are also described.

### FIELD OF THE INVENTION

The present invention is in general technical fields of functional synthetic peptides and uses therefor; synthetic peptide sequences; and computational methods for synthetic peptide sequence construction.

### BACKGROUND OF THE INVENTION

Amino acids (referred to herein from time to time as AAs) are small organic molecules that consist of an alpha (central) carbon atom linked to an amino group, a carboxyl group, a hydrogen atom, and a variable component called a side chain. The side chains of amino acids have different chemistries, with the largest group of amino acids having nonpolar side chains, others having side chains with positive or negative charges, and still others having polar but uncharged side chains. There are twenty naturally occurring amino acids, each bearing a chemically unique side chain, as well as numerous synthetic or non-naturally occurring amino acids which consist of an alpha (central) carbon atom linked to an amino group, a carboxyl group, a hydrogen atom, and a side chain that does not necessarily correspond to a naturally occurring amino acid.

A peptide encompasses organic molecules composed of amino acids (or amino acid residues), whether natural or synthetic, and linked together chemically by peptide bonds. The peptide bond involves a single covalent link between the a-carboxyl (oxygen-bearing carbon) of one amino acid and the amino nitrogen of a second amino acid. Small peptides with fewer than about ten constituent amino acids are typically called oligopeptides, and peptides with more than ten amino acids are termed polypeptides. Molecules with molecular weights of more than 10,000 Daltons (50-100 amino acids) are usually termed proteins. A more detailed discussion of peptides may be found in U.S. Pat. No. 7,739,055, the contents and disclosure of which are hereby incorporated herein by reference.

Peptides differ from each other through their amino acid composition and sequence, location, function, and spatial configuration. The chemistry of amino acid side chains is critical to protein structure because these side chains can bond with one another to hold a length of protein in a certain shape or conformation. Because of side chain interactions, the sequence and location of amino acids in a particular protein may guide where the bends and folds occur in that protein. These determine how the protein folds into a particular three-dimensional configuration, which, in turn, determines the activity and function of the protein.

In biological systems, peptides have varying functions. Some of them are structural materials (e.g., keratin) whereas others act as enzymes. Other functions include transport (e.g., hemoglobin), immunity (e.g., antibodies), and regulation. Proteins perform a vast array of functions within organisms including catalyzing metabolic reactions, DNA replication, responding to stimuli, providing structure to cells and organisms, and transporting molecules from one location to another. In one example, signal peptides are peptides present at the N-terminus (or occasionally C-terminus) of proteins, which can function to prompt a cell to translocate the protein, usually to a cellular membrane. Peptides are also known to mediate a variety of physiological responses in many organisms, including humans. Among these bioactive peptides are the peptide hormones, such as glucagon and insulin, which regulate glucose levels in the blood; gastrin and secretin, which control digestive processes; and follicle-stimulating hormone (FSH) and luteinizing hormone, which regulate reproductive processes. Other bioactive peptides act as growth factors, including somatotropin (growth hormone), erythropoietin, and NGF (nerve growth factor).

The wide scope and variety of these and other useful roles have led to interest in the computational design and generation of peptide sequences to support manufacture of synthetic peptides performing specific functions. A particular challenge in this effort is the navigation of the myriad of possible amino acid sequences and configurations. In one example, U.S. Pat. No. 7,739,055 describes methods to create databases of peptides having a desirable property involving analyzing a database of known peptides for a pattern statistically associated with the desirable property. The set of sequences being analyzed may include sequences of a desired length containing all or substantially all combinations of amino acids that conform to at least one of the set of patterns, and once the database is identified, the database may be processed in a pattern recognition procedure that identifies a set of patterns that may be representative of a peptide having the desirable property, with a set of newly generated peptide sequences processed to score these new sequences against the identified patterns to correlate the patterns to the sequences and determine a degree of association or similarity between one or more of the new sequences and the set of identified patterns. In another example, U.S. Pat. No. 7,228,238 describes the algorithmic methods of designing peptides or peptide analogue molecules employing certain derived properties from analyses of the target protein sequences, in addition to relevant distributions of amino acids, for weighted and constrained random assignments to the templates to produce the peptides. More specifically, Wu et al. (ACS Synthetic Biology 2020 9 (8), 2154-2161) have generally described a targeted method the generation of signal peptides by transforming a non-functional sequence into a functional sequence using attention-based neural networks that are not transparent as to their decision-making.

Despite these advances, an unmet need remains for a computational method for generating or constructing peptide sequences, both targeted and untargeted, which can, with tunable accuracy and speed, generate sequences that, when used in synthetic peptide synthesis, employ understandable and transparent computational techniques with a high probability of a desired functionality as exemplified in the cancer treatment method described in U.S. Published Patent Application No. 2020/0360469.

### SUMMARY OF THE INVENTION

In a first aspect, the present invention is directed to a computational method for constructing a synthetic peptide sequence. In this aspect, the method of the present invention includes the steps of (i) identifying a candidate sequence building block set comprising candidate sequence building blocks from a base set comprising known functional peptide sequences and optionally known non-functional peptide sequences; (ii) selecting a qualified sequence building block set comprising qualified sequence building blocks from said candidate sequence building block set; said qualified sequence building blocks satisfying a threshold requirement and (iii) assembling said qualified sequence building blocks to generate a synthetic peptide sequence.

In another aspect, the present invention is directed to a method for constructing a functional synthetic peptide. In this aspect, the method of the present invention includes the steps of (i) identifying a candidate sequence building block set comprising candidate sequence building blocks from a base set comprising known functional peptide sequences and optionally known non-functional peptide sequences; (ii) selecting a qualified sequence building block set comprising qualified sequence building blocks from said candidate sequence building block set; said qualified sequence building blocks satisfying a threshold requirement; (iii) assembling said qualified sequence building blocks to generate a synthetic peptide sequence and (iv) forming a functional synthetic peptide correlating to the synthetic peptide sequence. The phrase “correlating to” is intended to mean that the functional synthetic peptide includes an amino acid chain length and arrangement or assembly that matches at least a portion of the synthetic peptide sequence constructed by the method of the present invention.

In another aspect, the present invention is directed to a synthetic peptide sequence. The functional peptide sequence of the present invention is constructed according to a method including the steps of (i) identifying a candidate sequence building block set comprising candidate sequence building blocks from a base set comprising known functional peptide sequences and optionally known non-functional peptide sequences; (ii) selecting a qualified sequence building block set comprising qualified sequence building blocks from said candidate sequence building block set; said qualified sequence building blocks satisfying a threshold requirement and (iii) assembling said qualified sequence building blocks to generate a functional synthetic peptide sequence.

In another aspect, the present invention is directed to a functional synthetic peptide. The functional synthetic peptide of the present invention includes or comprises the synthetic peptide sequence of the present invention. The functional synthetic peptide of the present invention includes, is formed from or prepared using a synthetic peptide sequence constructed by a method that includes the steps of (i) identifying a candidate sequence building block set comprising candidate sequence building blocks from a base set comprising known functional peptide sequences and optionally known non-functional peptide sequences; (ii) selecting a qualified sequence building block set comprising qualified sequence building blocks from said candidate sequence building block set; said qualified sequence building blocks satisfying a threshold requirement; (iii) assembling said qualified sequence building blocks to generate a synthetic peptide sequence; and (iv) forming a functional synthetic peptide substantially correlating to the synthetic peptide sequence. The functional synthetic peptide of the present invention is formed by a method comprising the steps of (i) identifying a candidate sequence building block set comprising candidate sequence building blocks from a base set comprising known functional peptide sequences and optionally known non-functional peptide sequences; (ii) selecting a qualified sequence building block set comprising qualified sequence building blocks from said candidate sequence building block set; said qualified sequence building blocks satisfying a threshold requirement; (iii) assembling said qualified sequence building blocks to generate a synthetic peptide sequence; and (iv) forming a functional synthetic peptide corresponding to said synthetic peptide sequence.

The functional synthetic peptides of the present invention can be utilized alone or in combination with other peptides to evoke a desired type of action or activity. Further, the functional synthetic peptides of the present invention may exhibit pharmacological, biological, catalytic or other activity and be useful as a component of compositions suitable for wide variety of end-use applications in technical fields such as therapeutics, medicines, chemical synthesis, purification and the like. Accordingly, in yet another aspect, the present invention is directed to a composition that includes the functional synthetic peptide of the present invention.

Further aspects of the invention are as disclosed and claimed herein.

### BRIEF DESCRIPTION OF THE DRAWINGS

FIG. 1 is a general diagrammatic overview, created with BioRender.com, of the method of the present invention as exemplified in Example 1. The method takes a base set that includes signal peptide sequences (also referred to as SPs) and optionally non-SP base sequences and constructs a knowledge base of building blocks (also referred to as BBs). The BBs are defined in terms of m-step ordered pairs of AAs, where m represents the number of spaces between AAs. Within each bracket two AAs are represented, followed by two numbers where the first number represents the location (loc) of the second AA from the first AA, while the second number represents the frequency with which this pair occurs. Diamonds in varying shades of gray represent the five broad classifications of AAs. BBs are discovered for each location across the length of the base set. Novel synthetic signal peptides (also referred to as SSPs) are generated by selecting a BB for each location.

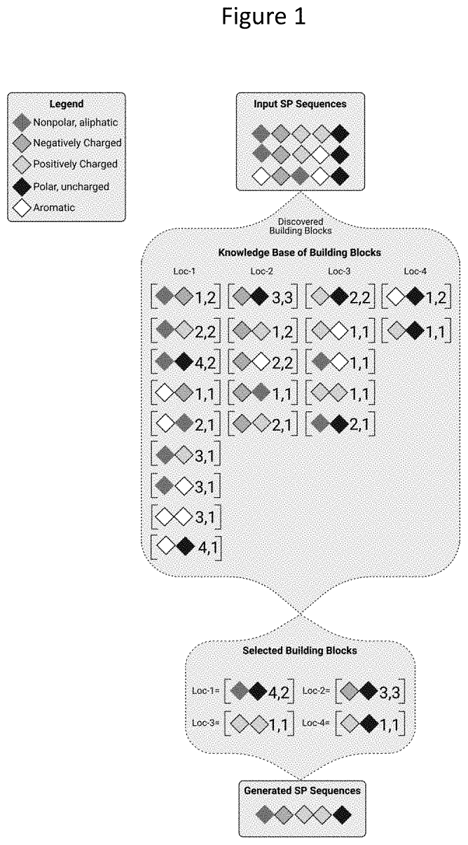

FIG. 2 is a graphical representation of the data in Table 1 from Example 1 describing accuracy assessment (5 replicates) for generated SSPs by rank-position range that were deemed SPs by Signal-BLAST.

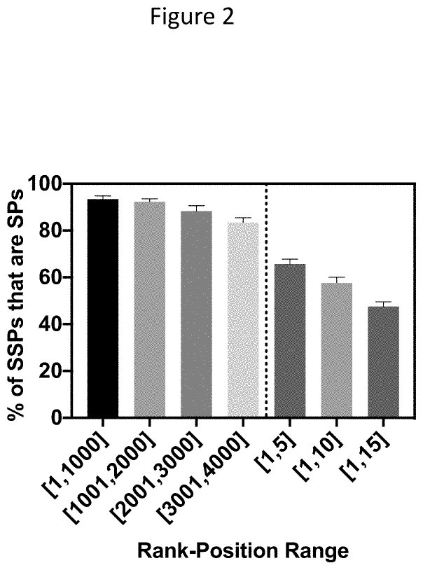

FIG. 3 is a graphical representation of the data in Table 3 from Example 1 displaying the entropy of single AAs (black) and bigrams (grey), which represent the complexity of the base data and the generated SSPs.

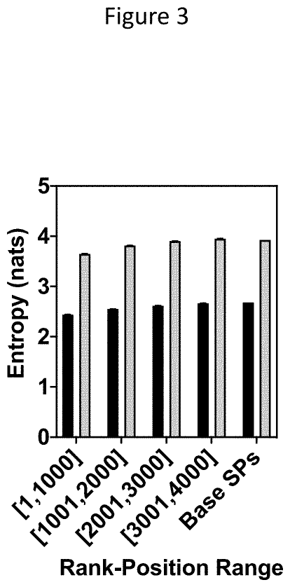

FIG. 4 is a general diagrammatic representation of the organism diversity of the base SPs (A) and the generated SSPs (B) from Example 1.

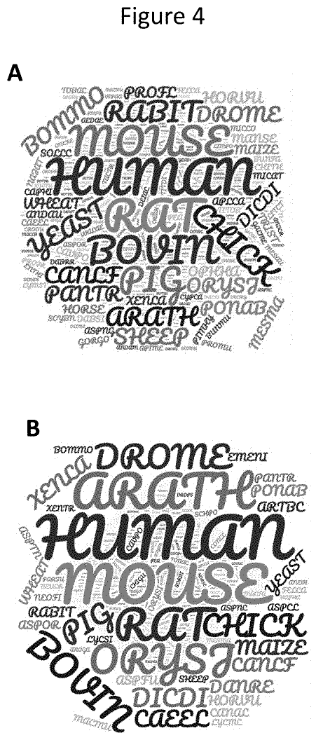

### DETAILED DESCRIPTION

For purposes of this application, the term “peptide” in singular, plural or possessive form is defined to include organic molecules composed of amino acids residues without regard to specific amino acid chain length. The term “peptide” therefore expressly includes peptides, oligopeptides, polypeptides and proteins as these terms are utilized in the art, for example as utilized in U.S. Pat. No. 7,739,055. One of ordinary skill in the art would appreciate that some overlap in specific chain length between various terms defining specific peptides may be expected, and deviations from these ranges do not in any way diminish the scope of the invention. For example, signal peptides are typically attached to or part of longer peptides such as proteins and function to prompt a cell to translocate the protein, often to a cellular membrane, and may in some cases be cleaved off from the larger non-signal peptide after translocation is complete.

For purposes of this application, the term “synthetic peptide” in singular, plural or possessive form is defined to include organic molecules composed of amino acid residues without regard to specific amino acid chain length that have amino acid sequences generated by a computational model that is either (a) not present in nature or (b) not the duplicate of any peptides provided as input to the computational method. For example, if the computational method provided ten signal peptide sequences, a generated synthetic peptide would not be a mirror image of any of those ten sequences. Additionally, peptides which are derived from naturally occurring peptides but which are modified by a computational method are also considered synthetic peptides.

The present invention is described herein with respect to various interrelated aspects and embodiments, including computational methods, peptide sequences and peptides. One of ordinary skill will understand and appreciate that elements or features used to describe one aspect or embodiment may be applicable and useful in describing other embodiments. Accordingly, descriptions and disclosure relating to elements or features of an aspect or embodiment of the present invention are hereby expressly relied on to describe and support those elements or features in other aspects or embodiments.

In a first aspect, the present invention is directed to a computational method for constructing a synthetic peptide sequence. The method of the present invention includes the steps of (i) identifying a candidate sequence building block set comprising candidate sequence building blocks from a base set comprising known functional peptide sequences and optionally known non-functional peptide sequences; (ii) selecting a qualified sequence building block set comprising qualified sequence building blocks from said candidate sequence building block set; said qualified sequence building blocks satisfying a threshold requirement and (iii) assembling said qualified sequence building blocks to generate a synthetic peptide sequence. In one or more embodiments, such as for example embodiments wherein the synthetic peptide sequence constructed by the present method is demonstrated, validated or otherwise verified to correlate or correspond to a functional synthetic peptide, the synthetic peptide sequence may be referred to as a functional synthetic peptide sequence.

In one or more embodiments, the base set includes known functional peptide sequences and optionally known non-functional peptide sequences. In one or more embodiments, the base set includes known functional peptide sequences and known non-functional peptide sequences.

In one or more embodiments, the known functional peptide sequences include signal peptide sequences; the known non-functional peptide sequences include non-signal peptide sequences; and the synthetic peptide sequence includes a synthetic signal peptide sequence. In one or more embodiments, the method of the present invention includes the steps of (i) identifying a candidate sequence building block set comprising candidate sequence building blocks from a base set comprising known signal peptide sequences and optionally known non-signal peptide sequences; (ii) selecting a qualified sequence building block set comprising qualified sequence building blocks from said candidate sequence building block set; said qualified sequence building blocks satisfying a threshold requirement and (iii) assembling said qualified sequence building blocks to generate a synthetic signal peptide sequence.

The method of the present invention includes the step of (i) identifying a set of candidate sequence building blocks from a base set of known functional peptide sequences and optionally non-functional peptide sequences. In a preferred embodiment, the base set may include functional peptide sequences and non-functional peptide sequences. In one or more embodiments, the functional peptide sequences are segregated from any non-functional peptide sequences in the set of candidate sequence building blocks and in such embodiments the identifying step (i) may further include segregating the set of candidate sequence building blocks into functional and non-functional subsets. Known functional peptide sequences are sequences known to include the specified functionality which the sequence to be constructed by the method is intended to possess. For example, when intending to construct a signal peptide sequence using the present method, known functional peptide sequences are those sequences correlating to peptides known to possess signal peptide functionality. Analogously, known non-functional peptide sequences are sequences known to lack the specified functionality which the sequence to be constructed by the method is intended to possess. For example, when intending to construct a signal peptide sequence using the present method, known non-functional peptide sequences are those sequences correlating to peptides known to lack signal peptide functionality.

More generally, the terms “functional” and “functionality” as used herein to describe peptide sequences and peptides that are intended to possess (as with sequences prior to verification) or possess (as with peptides) one or more specified activities, functions, properties or utilities. For example, we broadly define the term “functional peptide” as a peptide having a sequence with a desired property where one such property could be to serve a signaling or targeting role. Conversely, “non-functional” peptides do not possess the specified activities, functions, properties or utilities. In one or more embodiments, peptide sequences may be one or more of biologically functional or industrially functional. As used herein, biologically functional is intended to describe functionalities relating to human-based utilities or functionalities. In one example of functionality that may be categorized as biological, signal peptides are typically attached to or part of longer peptides such as proteins and function to prompt a cell to translocate the protein, often to a cellular membrane, and may in some cases be cleaved off from the larger non-signal peptide after translocation is complete. Industrial functionality is intended to describe to functionalities or utilities involving industrial or agricultural processes such as chemical manufacturing and processing, catalysis, purification and the like. One of ordinary skill will appreciate that, in some embodiments, there may be overlap in these categories such as when an industrially functional peptide uses a biological mechanism in performing its utility. It should also be understood that the “functional” and “functionality” is intended to include not only a singular functionality but also one or more functionalities.

Accordingly, in one or more embodiments or aspects herein, a functional synthetic peptide sequence may be a biologically functional peptide sequence and a functional synthetic peptide may be a biologically functional synthetic peptide. Further, in one or more embodiments or aspects herein, a functional synthetic peptide sequence may be an industrially functional peptide sequence or a functional synthetic peptide may be an industrially functional synthetic peptide.

In identifying step (i), a candidate sequence building block set including candidate sequence building blocks is identified or selected from the base set on the basis of their presence in or absence from the base set. In one or more embodiments, a sequence building block may be defined as an ordered pair of amino acids and a sequence building block set of may be defined as a collection of ordered pairs of amino acids. In this embodiment, a set of candidate sequence building blocks corresponds to a set of m-step ordered pairs of amino acids that occur at least one time in the functional or non-functional peptide sequences in the base set, wherein m represents the number of spaces between the amino acids of the ordered pair and the set of m-step ordered pairs forms a multi-layered vector space with m ranging from 1 to |S|−1 with |S| equal to the number of amino acids in the sequence.

Accordingly, in one or more embodiments, the identifying step (i) includes identifying a set of m-step ordered pairs of amino acids that occur at least one time in said base set, wherein m represents the number of spaces between said amino acids and the set of m-step ordered pairs forms a multi-layered vector space with m ranging from 1 to |S|−1 with |S| equal to the number of amino acids in the sequence.

In step (i) the candidate sequence building block set that includes candidate sequence building blocks is identified or selected from a base set. The base set utilized in this step includes known functional peptide sequences and optionally but preferably further includes known non-functional peptide sequences. The base set employed will depend upon a number of factors including for example the desired functionality or functionalities of the peptide sequence to be constructed by the method or the particular mechanism of action. The base set may be created, defined or chosen by one of skill in the art based on these factors.

Sources containing information from which a base set can be created will be known to one of ordinary skill in the art and may be publicly available commercially in electronically stored form. In the example of the present method set forth herein, the information for the base set was available from the Center of Biological Sequence Analysis at the Technical University of Denmark. Other examples of informational sources for the base set include without limitation, the University of Nebraska Medical Center Antimicrobial Peptide Database and Antimicrobial sequences database (AMSDb) (2002), which may be supplemented with additional peptides from Swiss-Prot/TrEMBL, A. Bairoch, R. Apweiler, Nucleic Acids Research 28, 45 (2002) AMSDb that is correlated to the SWISS-PROT protein sequences database., a database updated and maintained within the framework of the European “PANAD” (Peptides As Novel Antiinfective Drugs) Project (European 5th framework programme, project N° QLK2-CT-2000-00411).

The method of the present invention includes the step of (ii) selecting qualified sequence building blocks from the set of candidate sequence building blocks; said qualified sequence building blocks satisfying one or more threshold requirements. In one or more embodiments, a candidate building block may be selected or identified as a qualified building block if its frequency of occurrence in the base set exceeds a numeric threshold value. In one or more embodiments, a candidate building block may be selected or identified as a qualified building block if the absolute value of its frequency of occurrence in the known functional peptide sequences minus its frequency of occurrence in the known non-functional peptide sequences exceeds a numeric threshold value. The collection of qualified sequence building blocks that corresponds to the set of sequence building blocks whose frequency of occurrence in the functional peptide sequences of the base set exceeds a numeric threshold value may be referred to as the qualified sequence building blocks set. Accordingly, in one or more embodiments, the selecting step (ii) includes selecting qualified sequence building blocks from the set of candidate sequence building blocks based on the frequency of occurrence of the m-step ordered pairs corresponding to the building block across the base set with said frequency of occurrence being greater than a given threshold value.

An important feature of the present invention is that the selecting step (ii) is “tunable” in the sense that selection of a specific building block as a qualified building block from the base set of candidate sequence building blocks may be adjusted by choice of the threshold value used in the selection step. For example, choosing a relatively higher threshold value will translate to establishment of a possibly narrower qualified sequence building block set. Conversely, choosing a relatively lower threshold value will translate to establishment of a possibly broader qualified sequence building block set.

The method of the present invention includes the step of (iii) assembling said qualified building blocks to generate a functional peptide sequence. A synthetic computational peptide sequence may have a length of n amino acids (also referred to AAs) and locations s(n), with s ranging from 1 to n. By way of brief non-limiting example, referencing a target peptide sequence with 70 AAs, position 1 in the sequence would be labeled 1(70), position 2 as 2(70), position 3 as 3(70) and so on. In one or more embodiments, the assembling step (iii) includes assigning a qualified sequence building block from said qualified building block set to each location s(n) in the sequence. In one or more embodiments, assignment of a qualified sequence building block from the qualified building block set to a position in the sequence may be based on for example a greedy or non-greedy algorithm.

In one or more embodiments, the method of the present invention is performed using a variant of the Multilayer Vector System (MLVS) model. The MLVS model is described in Exploiting Multi-layered Vector Spaces for signal peptide detection, Int. J. Data Mining and Bioinformatics, Vol. 13, No. 2, 2015, hereby incorporated herein by reference, in regard to signal peptide detection. In one or more embodiments, exemplified below in regard to signal peptides, the MLVS model as utilized in the present invention comprises three primary steps: (i) discovery of candidate building blocks (also referred as BBs), (II) selection of candidate BBs satisfying a threshold requirement, labeled then as qualified BBs, and (iii) assembly of qualified BBs to create new synthetic signal peptide sequences. These steps are performed in the context of a base set of SP and non-SP sequences. First, m-step ordered pairs of AAs that occur at least one time in a base sequence are identified, where m represents the number of spaces between AAs. All the ordered pairs made up of consecutive AAs form the 1-step ordered pair, P₁. A MLVS is the set of m-step ordered pairs, P₁, P₂, . . . , Pₖ. By way of non-limiting example, the ASLGVsequence contains the 3-step ordered pair [S, V] where S represents the anchor AA occurring at location 2 and V represents the tail AA occurring 3 locations to the right of S. Informally, the number of m-step ordered pairs of AAs that can be derived from a set of protein sequences is a function of the length of the sequences and number of distinct AAs contained within those sequences.

In step (ii), a subset of candidate BBs, termed qualified BBs, may be identified. In one or more embodiments, candidate BBs are filtered to identify qualified BBs based on the difference in the normalized frequency of occurrence of an m-step ordered pair across the base set. The normalized frequency of a BB with respect to non-SP and SP sequences is equal to its frequency of occurrence divided by the number of non-SP and SP sequences, respectively. Those candidate BBs having an absolute difference value greater than a user-defined threshold between 0 and 1 are selected as qualified BBs. The resulting set of qualified BBs are those that occur frequently in the SP base sequences and infrequently in the non-SP sequences, and vice-versa, allowing for increased diversity.

In one or more embodiments, the assembling step (iii) includes assembling said qualified sequence building blocks in the absence of reference to a template sequence comprising at least one of (a) a sequence of randomly selected amino acids, (b) a known functional peptide sequence, (c) a known non-functional peptide sequence, and (d) a partially filled sequence with missing amino acids. This may be referred to as a de novo or “start from scratch” approach. In this assembling step (iii), qualified BBs are assembled to form a synthetic peptide sequence of length n. For one or more embodiments, in constructing a synthetic peptide sequence S of length n, denoted as (s(1), s(2), . . . , s(n)), each location s(i) (1≤i≤n) is assigned all qualified m-step ordered pairs of AAs where the upper bound on the step size m is (n−i). The qualified BBs at each location s(i) are sorted in non-decreasing order based on their frequency of occurrence across the base set. The frequency of a BB with a step size of m at location s(i) is the number of SP base sequences that contain the BB anchored at that location. Assembling a new synthetic peptide sequence starts with the selection of a BB at sequence location 1, followed by location 2, and terminating at location (n−1). BBs are selected at each location based on an input parameter, called the rank-position range, which represents a range of integer numbers from a specified lower to an upper bound. The rank-position value at a sequence location i was determined randomly. Once a BB of step size m is selected at location i, its inclusion in the SSP is determined by the following conditions: If an AA already exists at location i and it matches the anchor AA of the selected BB, then the tail AA is assigned to the location (i+m) if it is currently unoccupied. If the location i is unoccupied, then the anchor AA is assigned to location i and the tail AA is inserted at location (i+m), if the tail location is currently unoccupied. In all other cases, the anchor and/or tail AAs of the BB are not inserted into the evolving sequence.

In one or more embodiments, the assembling step (iii) includes assembling the qualified sequence building blocks with reference to one or more template sequences wherein said template sequences comprises at least one of (a) a sequence of randomly selected AAs, (b) a known functional peptide sequence, (c) a known non-functional peptide sequences, and (d) a partially filled sequence with missing AAs. The sequence is of size n. Given the template, each AA at location i, starting with 1=1, is replaced with the integer value representing the rank position of the highest-ranking matching BB at that location. Specifically, the BBs at sequence location i are continuing until either a BB is found that matches the anchor and tail AAs in the given template sequence or a user established out-of-rank threshold is reached. The out-of-rank range threshold improves the efficiency of the method by eliminating those BBs unlikely to have a positive impact on the transformation. If a matching BB is found within the out-of-rank threshold, then the anchor AA is replaced with the corresponding rank position value; otherwise, the anchor AA is replaced with −1. In the case of missing AAs, the position is automatically assigned a −1. Once all the AAs have been replaced with the rank position values, the next step is to replace the AAs at those sequence locations having either a −1, or a rank position value lower than another user given threshold (replacement threshold) but higher than the out-of-rank range threshold, with a randomly selected BB having a rank-position value within a specified range. Sequence locations are updated from left to right.

The MLVS model represents a synthetic peptide sequence S as a multi-layered collection of ordered m-step pairs (i,j)∈Σ, denoted by P_{m(i,j)}, m=1, 2, . . . , k. The parameter m stands for the number of spaces between the elements of the pair, downstream in the flow (left to right) of the sequence, and k is the maximum admissible value of m. The elements of an ordered pair (i,j) are referred to as the anchor and tail, respectively, and the location where the anchor element occurs in the sequence is referred to as the anchor location. The elements of the alphabet Σ are the AAs belonging to a protein sequence. Ordered pairs made up of consecutive elements of the sequence are said to form the family of 1-step pairs, P_{1/(i,j)}. Allowing multiple spaces between the elements of the ordered pair generates a multitude of m-step pairs (families) P₁, P₂, . . . , Pₘ, . . . , Pₖ, creating a multi-layered k-clustering Cₖ made up of sets P_{m/(i,j)}, m=1, 2, . . . , k

Cₖ=∪ₘ∪_{(i,j)}P_{m/(i,j)}

The binding factor between the elements of a particular set P_{m/(i,j)} is the step size m, common for all ordered pairs making up the family. The total number of ordered pairs that can be drawn from the alphabet is |Σ|² and the maximum size of Cₖ is reached for k=|S|−1, where the maximum m-step ordered pair (m=k=|S|−1) spans the entire sequence. Hence, a sequence, S, can is represented as the union of all such ordered pairs at k distinct layers.

In the context of generating synthetic signal peptide sequences, two distinct families of m-step pairs P₁, P₂, Pₘ, . . . , Pₖ, are created; one family, P₁, P₂, . . . , Pₘ, . . . , Pₖ, with respect to the base SP sequences and another family, P′₁, P′₂, . . . , P′ₘ, . . . , P′ₖ, with respect to the base non-SP sequences. The candidate BBs are identified based on the absolute difference in the frequency of occurrence of individual m-step ordered pairs of AAs (i,j)∈Σ between corresponding sets Pₘ and P′ₘ, for m=1, 2, . . . , k. The selection of qualified BBs from the candidate BBs and the subsequent process of assembling new synthetic signal peptide sequences may follow the steps described above in one or more embodiments.

In one or more embodiments, the method of the present invention may involve single AAs as BBs instead of m-step ordered pairs of AAs as described above. In such embodiments, the twenty canonical naturally-occurring AAs (i.e., BBs) may be sorted in non-decreasing order based on their frequency of occurrence in the given base sequences at each location, (s1, s2, . . . , s(n). Assembling a new synthetic signal peptide sequence may start with the selection of a BB at sequence location 1, followed by location 2, and terminating at location (n−1) wherein n is the number of amino acids in the sequence. BBs are selected at each location based on an input parameter, called the rank-position range, which represents a range of integer numbers from a specified lower to an upper bound. In some embodiments, such as those involving biological sets, the upper bound typically would not exceed 20. The rank-position range value selected at a given location i determines the BB (i.e., AA) at that location. One of ordinary skill will appreciate that the method could be applied to alphabets beyond the twenty canonical AA, adjusting the upper bound to match the number of AA in an expanded alphabet.

The functional peptide sequence generated by the above method may be useful in forming a functional synthetic peptide. In another aspect, then, the present invention is directed to a method for forming a functional synthetic peptide. In this aspect, the method of the present invention includes the steps of (i) identifying a set of candidate sequence building blocks from a base set including known functional peptide sequences and optionally non-functional peptide sequences; (ii) selecting qualified sequence building blocks from said set of candidate sequence building blocks; said qualified building blocks satisfying a threshold requirement; (iii) assembling said qualified sequence building blocks to generate a functional synthetic peptide sequence; and (iv) forming a functional synthetic peptide substantially correlating to said functional peptide sequence. In one or more embodiments, a functional synthetic peptide sequence and/or a functional synthetic peptide is selected from the group consisting of a biologically functional peptide sequence. In one or more embodiments, a biologically functional synthetic peptide includes a synthetic signal peptide.

The forming step (iv) in the above method may be performed using known and conventional techniques for synthetic peptide synthesis. The functional synthetic peptides of the present invention can be synthesized in solution or on a solid support in accordance with conventional techniques. Various automatic synthesizers are also commercially available and can be used in accordance with known protocols. See, for example, Stewart and Young, Solid Phase Peptide Synthesis, 2d. ed., Pierce Chemical Co., (1984); Tam et al., J. Am. Chem. Soc. 105:6442, 1983; Merrifield, Science 232:341-347, 1986; and Barany and Merrifield, The Peptides, Gross and Meienhofer, eds, Academic Press, New York, 1 284; Barany et al., Int. J. Peptide Protein Res. 30:705-739, 1987; and U.S. Pat. No. 5,424,398, each incorporated herein by reference. Stepwise elongation, in which the amino acids are connected step-by-step in turn, may also be useful.

In another aspect, the present invention is directed to a synthetic peptide sequence. The computational synthetic peptide sequence of the present invention is constructed according to a method including the steps of (i) identifying a set of candidate sequence building blocks from a base set of known functional peptide sequences and non-functional peptide sequences; (ii) selecting qualified sequence building blocks from said set of candidate sequence building blocks; said qualified building blocks satisfying a given threshold requirement and (iii) assembling said qualified sequence building blocks to generate a synthetic signal peptide sequence.

In another aspect, the present invention is directed to a functional synthetic peptide. The biologically functional synthetic peptide of the present invention includes, is formed from or prepared using a synthetic peptide sequence constructed by a method that includes the steps of (i) identifying a set of candidate sequence building blocks from a base set of known functional peptide sequences and non-functional peptide sequences; (ii) selecting qualified sequence building blocks from said set of candidate sequence building blocks; said qualified sequence building blocks satisfying a given threshold requirement and (iii) assembling said qualified sequence building blocks to generate a synthetic signal peptide sequence.

The functional synthetic peptides of the present invention may be useful, typically as a component of a composition, in a variety of end-use applications including but not limited to pharmaceuticals, agriculture, food additives, textiles, fragrances, and biofuels, and synthetic materials. In one or more embodiments, the functional synthetic peptide may be a component of a composition. Accordingly, in another aspect, the present invention is directed to a composition comprising the functional synthetic peptide of the present invention. The present invention may be directed to a composition comprising a functional synthetic peptide wherein the functional synthetic peptide is formed by a method that includes the steps of (i) identifying a candidate sequence building block set comprising candidate sequence building blocks from a base set comprising known functional peptide sequences and optionally known non-functional peptide sequences; (ii) selecting a qualified sequence building block set comprising qualified sequence building blocks from said candidate sequence building block set; said qualified sequence building blocks satisfying a threshold requirement; (iii) assembling said qualified sequence building blocks to generate a synthetic peptide sequence and (iv) forming a functional synthetic peptide correlating to the synthetic peptide sequence. In one or more embodiments, the composition comprises a functional synthetic peptide wherein the functional synthetic peptide includes, is formed from or is prepared using a synthetic peptide sequence constructed by a method that includes the steps of (i) identifying a candidate sequence building block set comprising candidate sequence building blocks from a base set comprising known functional peptide sequences and optionally known non-functional peptide sequences; (ii) selecting a qualified sequence building block set comprising qualified sequence building blocks from said candidate sequence building block set; said qualified sequence building blocks satisfying a threshold requirement; and (iii) assembling said qualified sequence building blocks to generate a synthetic peptide sequence.

The compositions of the present invention may include other components or ingredients, which facilitate or support the intended use of the composition. Non-limiting examples include one or more of the groups consisting of other active ingredients, carriers, diluents, solvents, dispersants, preservatives, thickeners and the like. In one or more embodiments, the compositions may include other peptides, the function of which is enhanced by the functional synthetic peptide of the present invention. For example, the functional synthetic peptide could act as a signal sequence, which could be appended to a known peptide such as insulin. The synthetic peptide would enhance the ability to produce insulin synthetically. In this way the functional synthetic peptide can be combined with other components or ingredients to form a useful composition.

The compositions of the present invention will include the synthetic signal peptides of the present invention in an effective amount, wherein an “effective amount” is generally intended to include amounts suitable for achieving the intended use or function of the composition. The effective amount may vary based on a number of factors such as the intended use, means of administration or application, identity and amount of other components or ingredients and the like.

Compositions of the present invention include but are not limited to biological compositions and industrial compositions. Biological compositions are generally intended to include compositions useful for administration to or use in conjunction with humans and may include medicinal compositions, therapeutics, vaccines, disease treatments, antimicrobials, enzyme inhibitors, biomarkers, biosensors, imaging tools and antibody production and the like. Examples of biological compositions and methods for their manufacture are described in U.S. Pat. Nos. 6,107,021; 7,399,825; 7,264,808; 8,841,420 and 9,663,557, the contents and disclosure of which are hereby expressly incorporated herein by reference. Industrial compositions are generally intended to include compositions useful in chemical processes such as synthesis, manufacturing, purification and the like as well as in agriculture, food additives, textiles, fragrances, biofuels and the like. Examples of industrial compositions utilizing signal peptide sequences are described in PCT Published Application W02016080672A1 and U.S. Pat. Nos. 10,351,816 and 10,975,363, the contents and disclosure of which are hereby expressly incorporated herein by reference.

### EXAMPLE 1

To demonstrate the method of the present invention as applied to synthetic peptide sequences for corresponding synthetic signal peptides, a base set of sequences was procured from a website maintained by the Center for Biological Sequence Analysis at the Technical University of Denmark. Specifically, the base set included 2,311 and 7,384 eukaryotic signal and non-signal sequences, respectively, with each sequence consisting of 70 AAs. A software tool available in the art under the name MULocDeep, identified 98% of the base signal peptide sequences as being secreted to the extracellular space.

Candidate BBs were identified from the base set using the MLVS model using a single occurrence as the threshold value. A total of 27,213 qualifying BBs were identified from the set of candidate BBs based on an absolute difference value greater than zero. A set of sequences from these BBs were then generated using rank-position ranges [1,1000], [1001,2000], [2001,3000], and [3001,4000]. Within each range, 500 sequences of 70 AAs were generated. For each range, the generated sequences were assessed for signal peptide functionality over 5 separate experiments using a software tool available in the art under the name Signal-BLAST, which identifies signal peptides based on direct comparison with a database of known proteins. Accuracy of this method is reflected by the percentage of generated sequences that were defined as signal peptide sequences by Signal-BLAST, which are set forth in Table 1 below.

TABLE 1
MLVS-Approach

| | [1, 1000] | [1001, 2000] | [2001, 3000] | [3001, 4000] |
|---|---|---|---|---|
| Run-1 | 92.4% | 91.8% | 89.6% | 84.6% |
| Run-2 | 95.0% | 92.0% | 88.6% | 81.8% |
| Run-3 | 93.8% | 93.0% | 91.0% | 86.2% |
| Run-4 | 91.6% | 90.8% | 87.4% | 81.2% |
| Run-5 | 94.2% | 94.0% | 85.0% | 83.4% |
| Avg | 93.4% | 92.3% | 88.3% | 83.4% |

In addition, the generated sequences were evaluated using the MULocDeep tool which identified 98% of the SSPs as being secreted to the extracellular space; this mimics the percentage found for the base signal peptide sequences.

The accuracy results of a naive method for generating SSPs that uses single AAs as BBs as opposed to the ordered pairs of the MLVS model are set forth in Table 2. The overall accuracy of this alternative method was lower and the number of synthetic signal peptide sequences generated was smaller in comparison to data generated using the MLVS model.

TABLE 2
BaseLine Method

| | Accuracy |
|---|---|
| Rank-Position Range | (Signal-Blast) (%) |
| [1, 5] | 64.40 |
| [1, 10] | 57.40 |
| [1, 15] | 47.20 |

Entropy for the sequences was also evaluated in each rank-position using the standard Shannon entropy measure for unigrams and bigrams, exemplified in U.S. Pat. No. 11,048,608, the contents and disclosure of which are incorporated herein by reference (reproduced in part here as Equation 1, changing “log” from the cited work to “In” to clarify that we chose the natural logarithm for this work),

H(P)=−ΣᵢPᵢln Pᵢ (1)

In this work the natural logarithm was chosen, but different base logarithms could be used and results would differ by a constant value. To apply the equation to sequences of AA, P(i) is taken to be the number of occurrences of a specific AA observed in a sequence divided by the total number of AA in the sequence, and the sum of all P(i) within a sequence must equal one. For unigrams, all individual AA (typically in the 20 AA alphabet, although alternate AA alphabets could be used), are taken as the alphabet. For bigrams, all permutations of two sequential AA are taken from the alphabet (20*20=400 permutations). As a concrete example, take the peptide sequence “AACTT”. The unigram frequencies are A(⅖), C(⅕), T(⅖), and substituting into the entropy equation yields

$$H ( P ) = - ( ( \frac{2}{5} ln ( \frac{2}{5} ) ) + ( \frac{1}{5} ln ( \frac{1}{5} ) ) + ( \frac{2}{5} ln ( \frac{2}{5} ) ) ) = 1.05 nats .$$

The bigram frequencies are AA(⅕), AC(⅕), CT(⅕), TT(⅕), and substituting into the equation yields

$$H ( P ) = - ( ( \frac{1}{5} ln ( \frac{1}{5} ) ) + ( \frac{1}{5} ln ( \frac{1}{5} ) ) + ( \frac{1}{5} ln ( \frac{1}{5} ) ) + ( \frac{1}{5} ln ( \frac{1}{5} ) ) ) = 1.29 nats .$$

Entropy values of SSPs were plotted for the 5 runs displayed as mean±standard deviation of the means (Table 3), with values for base sequences included for comparison. Each rank position displayed low standard deviation, demonstrating batch consistency. The data in Table 3 below demonstrates that entropy increases as the rank-position value increases for unigrams and bigrams, with the [3001,4000] set having similar entropy to the base sequences. As the randomness of sequences appeared to be a function of the rank-position parameter, it was reasonable to conclude that a user could “tune” this parameter to optimize accuracy at the expense of sequence randomness and novelty.

TABLE 3
Entropy Statistics

| | # Residues | # Runs | Mean (nats) | std |
|---|---|---|---|---|
| 1-1000 | 1 | 5 | 2.439357 | 0.004858 |
| | 2 | 5 | 3.649838 | 0.005875 |
| 1001-2000 | 1 | 5 | 2.550365 | 0.003772 |
| | 2 | 5 | 3.816099 | 0.003658 |
| 2001-3000 | 1 | 5 | 2.617792 | 0.003151 |
| | 2 | 5 | 3.903525 | 0.003077 |
| 3001-4000 | 1 | 5 | 2.665958 | 0.003725 |
| | 2 | 5 | 3.951362 | 0.005157 |
| Base-Seqs | 1 | 1 | 2.682886 | N/A |
| | 2 | 1 | 3.925006 | N/A |

An additional 9,555 sequences were generated using a rank-position range of [1, 3000]. According to an evaluation using Signal-BLAST, a total of 8,444 (88.37%) of the sequences were identified as signal peptide sequences covered a diverse range of organisms similar to that of the base set signal peptide sequences.

Lastly, an experiment was conducted to evaluate the accuracy in converting non-signal sequences into SSPs. Unlike the previous experiments, in which a template sequence was not referenced, (a “blank slate” or de novo approach), the assembling step for this experiment included reference to 7,384 non-signaling eukaryotic sequences with each of the 7,384 sequences individually used as a template where only those AAs not falling into the user defined range where changed. Three different rank ranges were used, [1,1000], [1,2000], and [1,3000], along with an out-of-rank range value of 6000. Each transformed sequence was verified using a software tool available in the art under the name SignalP-5.0. The results are set forth in Table 4 below. As shown in Table 4, there is a tradeoff between the accuracy and percentage of AAs modified during the transformation process. Using a smaller rank-position range resulted in higher probability of achieving a successful transformation. However, smaller ranges also resulted in a higher percentage of modified AAs during the transformation process.

TABLE 4

| Rank | | |
|---|---|---|
| Position | | Average % |
| Range | Accuracy | Modified AAs |
| [1, 1000] | 96.12% | 53.40% |
| [1, 2000] | 72.75% | 31.69% |
| [1, 3000] | 47.14% | 27.20% |

The foregoing description of various embodiments of the invention has been presented for purposes of illustration and description. It is not intended to be exhaustive or to limit the invention to the precise embodiments disclosed. Numerous modifications or variations are possible in light of the above teachings. The embodiments discussed were chosen and described to provide the best illustration of the principles of the invention and its practical application to thereby enable one of ordinary skill in the art to utilize the invention in various embodiments and with various modifications as are suited to the particular use contemplated. All such modifications and variations are within the scope of the invention as determined by the appended claims when interpreted in accordance with the breadth to which they are fairly, legally, and equitably entitled.

That which is claimed is:

1. A computational method for constructing a synthetic peptide sequence, said method comprising the steps of (i) identifying a candidate sequence building block set comprising candidate sequence building blocks from a base set comprising known functional peptide sequences and optionally known non-functional peptide sequences; (ii) selecting a qualified sequence building block set comprising qualified sequence building blocks from said candidate sequence building block set; said qualified sequence building blocks satisfying a threshold requirement and (iii) assembling said qualified sequence building blocks to generate a synthetic peptide sequence.

2. The computational method of claim 1 wherein said identifying step (i) includes identifying a set of m-step ordered pairs of amino acids that occur at least one time in said base set, wherein m represents the number of spaces between said amino acids and the set of m-step ordered pairs forms a multi-layered vector space with m ranging from 1 to |S|−1 with |S| equal to the number of amino acids in the sequence.

3. The computational method of claim 2 wherein said selecting step (ii) comprises selecting said qualified sequence building blocks from said candidate sequence building block set based on the frequency of occurrence of said m-step ordered pairs, with said frequency of occurrence being greater than a given threshold value.

4. The computational method of claim 3 wherein said synthetic peptide sequence comprises a length of n amino acids located along the sequence at locations s(n) and said assembling step (iii) includes assigning a qualified sequence building block from said qualified building block set to each said location s(n) in said sequence.

5. The computational method of claim 1 wherein said assembling step (iii) comprises assembling said qualified sequence building blocks in the absence of reference to a template sequence comprising at least one of (a) a sequence of randomly selected amino acids, (b) a known functional peptide sequence, (c) a known non-functional peptide sequence, and (d) a partially filled sequence with missing amino acids.

6. The computational method of claim 1 wherein said assembling step (iii) includes assembling said qualified sequence with reference to one or more template sequences wherein said template sequences comprises at least one of (a) a sequence of randomly selected amino acids, (b) a known functional peptide sequence, (c) a known non-functional peptide sequences, and (d) a partially filled sequence with missing amino acids.

7. A method for forming a functional synthetic peptide, said method comprising the steps of (i) identifying a candidate sequence building block set comprising candidate sequence building blocks from a base set comprising known functional peptide sequences and optionally known non-functional peptide sequences; (ii) selecting a qualified sequence building block set comprising qualified sequence building blocks from said candidate sequence building block set; said qualified sequence building blocks satisfying a threshold requirement and (iii) assembling said qualified sequence building blocks to generate a synthetic peptide sequence and (iv) forming a functional synthetic peptide correlating to the synthetic peptide sequence.

8. The method of claim 7 wherein said functional synthetic peptide is a synthetic signal peptide.

9. A synthetic peptide sequence constructed according to the method of claim 1.

10. A functional synthetic peptide comprising, formed from or prepared using a synthetic peptide sequence constructed according to the method of claim 1.

11. A functional synthetic peptide comprising, formed from or prepared using the synthetic peptide sequence of claim 9.

12. The functional synthetic peptide of claim 11 wherein said functional synthetic peptide is a synthetic signal peptide.

13. A composition comprising an effective amount of the functional synthetic peptide of claim 10.

14. A composition comprising an effective amount of the functional synthetic peptide of claim 12.

15. The composition of claim 13 wherein said composition is selected from the group consisting of a biological composition and an industrial composition.

16. The composition of claim 14 wherein said composition is selected from the group consisting of a biological composition and an industrial composition

17. The composition of claim 13 further comprising one or more component selected from the group consisting of carriers, diluents, solvents, dispersants, preservatives, thickeners and other active ingredients.

18. The composition of claim 14 further comprising one or more component selected from the group consisting of carriers, diluents, solvents, dispersants, preservatives, thickeners and other active ingredients.
